(19) United States
(12) Patent Application Publication
(10) Pub. No.: US 2018/0190029 A1
(43) Pub. Date: Jul. 5, 2018

(54) HEAD MOUNTED COMBINATION FOR INDUSTRIAL SAFETY AND GUIDANCE

(71) Applicant: HONEYWELL INTERNATIONAL INC., MORRIS PLAINS, NJ (US)

(72) Inventors: ROD STEIN, EDMONTON (CA); COLIN GREGORY PEART, EDMONTON (CA)

(21) Appl. No.: 15/399,481

(22) Filed: Jan. 5, 2017

(51) Int. Cl. G06T 19/00; G06F 3/01; G06T 19/20; A61F 9/02; A42B 3/18; A42B 3/22

(52) U.S. Cl. CPC G06T 19/006; G06F 3/012; G06F 3/013; G06T 2219/004; A61F 9/029; A42B 3/185; A42B 3/225; G06T 19/20

(57) ABSTRACT

A head mounted combination for use in an industrial facility includes an eye shield and an augmented reality headset computer system for communicating over a wireless channel including a processor, system memory, transceiver, a location, orientation and a gaze sensor. A display(s) is embedded in or on an inside surface of the eye shield or lens and coupled to the processor. Client software stored in the system memory determines what the user is looking at together with a 3D model of system elements in the industrial facility used for overlaying computer generated representations of viewed system elements within the user's field of view. Display marker(s) is added to the viewed system elements which have further data available to indicate availability. Responsive to the user triggering the display marker, the first element data is displayed in the display for viewing by the user together with the real world view.

### FIELD

Disclosed embodiments relate to augmented reality in industrial applications.

### BACKGROUND

Augmented reality (AR) is a live view of a physical environment whose elements are augmented by computer-generated sensory inputs, for example, video, sound, graphics or Global Positioning System (GPS) data. AR is commonly integrated with a head mounted display (HMD) device which is a device worn on the head of the user or as part of a helmet, that has a relatively small display optic in front of one (monocular HMD) or in front of each eye (binocular HMD). The AR HMD combines computer-generated imagery (CGI) with live imagery from the real world.

### SUMMARY

This Summary is provided to introduce a brief selection of disclosed concepts in a simplified form that are further described below in the Detailed Description including the drawings provided. This Summary is not intended to limit the claimed subject matter's scope.

Disclosed embodiments recognize known head mounted safety equipment, such as safety glasses, face shields, hard hats and respirators, provide only their intended safety function. Disclosed embodiments add significant functionality to the head mounted safety equipment that assists the user by providing a head mounted combination including (i) head mounted safety equipment including at least an eye shield or lenses as well as (ii) an AR headset computer system (ARHCS) including a processor and a transceiver for supporting wireless communications, and at least one display (generally a pair of displays, one for each eye) coupled to the processor embedded in or on an inside surface of an eye shield or the lenses. Using a disclosed head mounted combination in an industrial work area has significant benefits including allowing the user the safety and productivity of normal visual sight while adding helpful process information to be brought into the visual (and optionally also auditory) field of the user to enhance the user's work experience.

### BRIEF DESCRIPTION OF THE DRAWINGS

FIG. 1 is a flow chart that shows steps in a method of protecting and assisting a user using a disclosed head mounted combination including at least an eye shield and an ARHCS, according to an example embodiment.

FIG. 2 is a block diagram representation of an example ARHCS, according to an example embodiment.

FIG. 3A shows an example head mounted combination comprising safety glasses including an example ARHCS.

FIG. 3B shows an example head mounted combination comprising a face shield including an example ARHCS.

FIG. 3C shows an example head mounted combination comprising a hard hat including an example ARHCS.

FIG. 3D shows an example head mounted combination comprising a respirator including an example ARHCS.

FIG. 3E shows an example head mounted combination comprising a screen resembling a contact lens including an example ARHCS shown adapted to be worn in contact with an eye of the user.

### DETAILED DESCRIPTION

Disclosed embodiments are described with reference to the attached figures, wherein like reference numerals are used throughout the figures to designate similar or equivalent elements. The figures are not drawn to scale and they are provided merely to illustrate certain disclosed aspects. Several disclosed aspects are described below with reference to example applications for illustration. It should be understood that numerous specific details, relationships, and methods are set forth to provide a full understanding of the disclosed embodiments.

One having ordinary skill in the relevant art, however, will readily recognize that the subject matter disclosed herein can be practiced without one or more of the specific details or with other methods. In other instances, well-known structures or operations are not shown in detail to avoid obscuring certain aspects. This Disclosure is not limited by the illustrated ordering of acts or events, as some acts may occur in different orders and/or concurrently with other acts or events. Furthermore, not all illustrated acts or events are required to implement a methodology in accordance with the embodiments disclosed herein.

Also, the terms “coupled to” or “couples with” (and the like) as used herein without further qualification are intended to describe either an indirect or direct electrical connection. Thus, if a first device “couples” to a second device, that connection can be through a direct electrical connection where there are only parasitics in the pathway, or through an indirect electrical connection via intervening items including other devices and connections. For indirect coupling, the intervening item generally does not modify the information of a signal but may adjust its current level, voltage level, and/or power level.

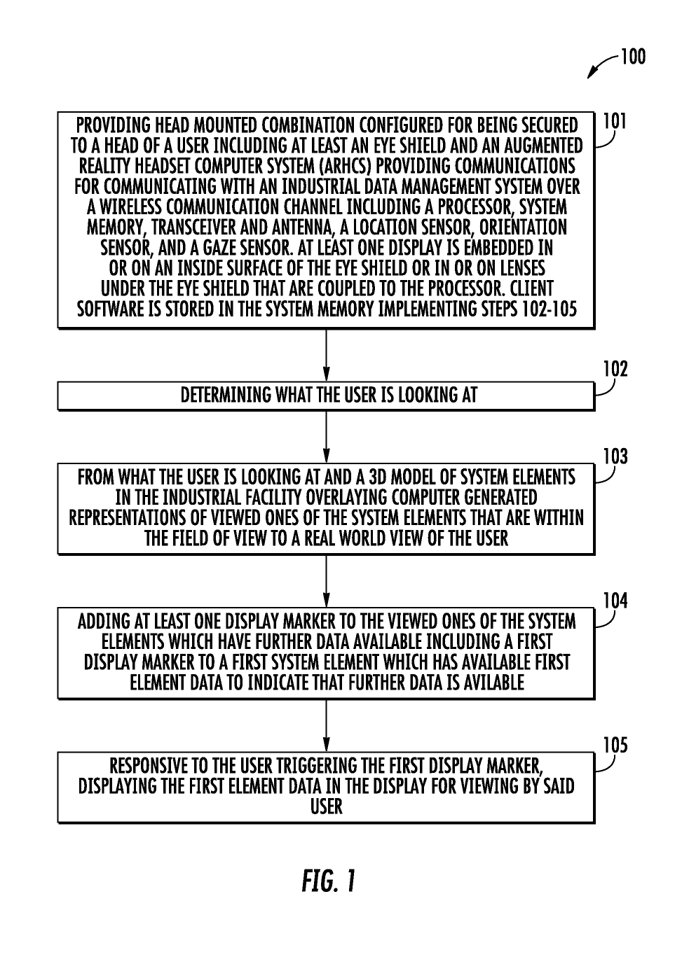

FIG. 1 is a flow chart that shows steps in a method 100 of protecting and assisting a user in an industrial facility using a disclosed head mounted combination, according to an example embodiment. An industrial facility is often managed using process control systems also known as control and instrumentation (C&I) systems. The industrial facility can include, for example, manufacturing plants, chemical plants, crude oil refineries, ore processing plants, paper plants or pulp plants.

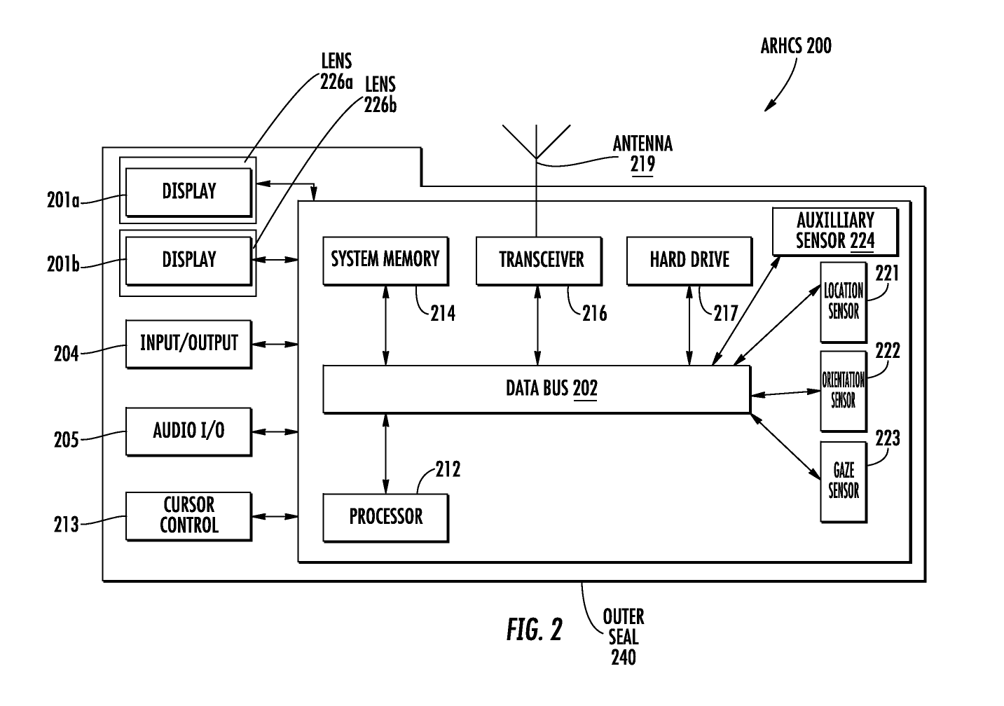

Step 101 comprises providing a head mounted combination configured for being secured to a head of the user including head mounted safety equipment including at least an eye shield and an ARHCS. The eye shield can be part of a helmet, safety glasses, face shield, self-contained breathing apparatus, or full face mask filter-based respirator. The eye shield can be also be removable. The ARHCS (see ARHCS 200 in FIG. 2 described below) includes a processor, system memory, and transceiver coupled to an antenna for providing wireless communications for bidirectionally communicating with an industrial data management system (e.g., server) over a wireless communication channel. The head mounted combination including a disclosed ARHCS which can be designed for use in hazardous environments and under industrial conditions.

The ARHCS also includes a location sensor, orientation sensor, a gaze sensor, and a single or pair of displays embedded in or on an inside surface of the eye shield or in or on lenses under the eye shield that are coupled to the processor, or embedded in or on the eye shield. The display(s) are coupled to the processor. Location and orientation sensors provide AR functionality. The gaze sensor is not always needed as the ARHCS can be functional without the gaze sensor if an alternate input device is provided to provide data entry, acknowledgment of communication, or user interface navigation. For example, key presses or cursor movements can be used to move a cursor to select and activate interactive elements, such as a physical device including a sleeve, chest, or helmet mounted buttons, or an input such as gesturing with the hands as an input.

Besides communications with the industrial data management system the ARHCS may also respond to requests initiated from external systems. Beyond requests from the server for data about the user, their condition, or the condition of the user's equipment, requests can be for simple task items including having the day's work tasks listed in the display and updating that from the control center, or other similar information. This also includes peer-to-peer information for workers working together on a task beyond the normal talking or line of sight communication. Client software is stored in the system memory that implements steps 102 to 105 described below. Some data can be locally stored in the memory of the ARHCS such as emergency evacuation routing information, hazard locations. This data can be operable in case of an emergency or a database disconnection.

Step 102 comprises determining what the user is currently looking at. What the user is looking at can be determined from a location of the user, a field of view of the user, and a gaze of the user, where this information being provided by the location sensor, orientation sensor, and a gaze sensor. What the user is looking at can also be calculated based on the position of the eye in relation to the displays and the user's theoretical field of view and location/orientation of the displays. What the user is looking at determines which interactive elements are to be projected in the display(s) into the head mounted combination's user's view (step 103 described below).

The gaze sensor can comprise cameras or other optical sensors that monitor the user's eyes to determine where the user is looking within their field of view. Markers in the plant on the processing equipment can also be used to technically adjust AR overlays to be aligned with the real physical field of view (to stop motion sickness type side effects). Combined with the location (from the location sensor) and orientation from the orientation sensor of the ARHCS, this information can be mathematically projected into a 3D model of the industrial facility (e.g., plant) to estimate in essentially real-time what the user is currently looking at in the real world.

Step 103 comprises from what the user is looking at and from a 3D database model of system elements in the industrial facility, computer generated representations system elements are overlaid in the display(s) that are within the field of view to a real world view of the user. A predetermined distance is a good example of a possible input for deciding if something should be shown. For example, the user can be close enough to something to see its AR overlays, despite the fact the physical system is located on the other side of a wall. The physical distance to hazards can automatically produce a warning sound and/or a display alert. Alerts can be anything from a flashing symbol or the object itself being highlighted. The database can be held by a memory of the ARHCS or be remotely accessed by the ARHCS. Thus gestural or gaze tracking is used for controlling when computer generated system representations and/or data is displayed in full.

What the user is looking at (determined in step 102) can also involve which interactive elements within the field of view the user is interested in interacting, with the gaze sensor determining where the user's eyes are pointed relative to the display, and that in turn is used to manage which projected elements are shown (step 103). The system can potentially use a physical peripheral such as a joystick with buttons, or a keypad to select the interactive elements without the use of the gaze sensor, analogous to tabbing through fields on a form. A peripheral controller can also be used in combination with the gaze sensor. For example, the user can look at an interactive element to highlight it, but then press a button on the peripheral controller to confirm that they want that interactive element to expand and provide more detail, or to trigger an action.

Step 104 comprises adding at least one display marker to indicate that further data is available to viewed ones of the system elements in the display(s) which have further data available including a first display marker to a first system element which has available first element data. For example, the display markers can comprise an icon or shape outlining the item marking it with a glowing outline that is not opaque.

Step 105 comprises responsive to the user triggering the first display marker, displaying the first element data in the display for viewing by the user. Regarding triggering, as noted above gaze detection can be used. It is recognized normally human eyes flicker over a significantly wide area. The processor can use sensed data obtained from the gaze sensor to identify when the user is intentionally looking at a particular marker or tag affixed to the plant equipment for a predetermined period of time. This can be used as part of a pure gaze detection system where after a certain time of focusing on the marker or tag, the display(s) are triggered. Triggering can also be accomplished manually with a button or touch detector mounted on the side of the ARHCS or in wired or wireless hand held device providing one or more controls including buttons, knobs, or joysticks that communicate with the ARHCS to act as a user interface device.

Significant elements of interaction with an AR display include the user being able to see what items they can interact with at any given time. The available items are selected based on what is in the user's field of view. The user is able to register an interest in any one particular object within their field of view. A temporary interest or potential interest shown in an object can change the visual display to highlight the object and set it apart visually from the rest of the available objects in the display. A limited amount of information such as the object's name or identifier can be drawn on the screen to allow a user to know which item they are looking at.

The user can also indicate an extended interest in an object that has been selected by the temporary interest system. When an extended interest is indicated, the ARHCS can respond by showing more detailed information about the object, and offering the user the ability to interact with the object. Finally, the user can indicate that they wish to take an action on an object that has been selected. Using a similar process as above, when the selected object is shown in the ‘extended interest’ state, sub-elements can be shown that the user can now select and activate.

The head mounted combination can be used in an industrial setting for a wide variety of purposes. For example, the element data can comprise real-time process data provided via wireless communication channels (e.g., from a server) that is projected in the display(s) on top of the user's visual field. In one particular example, a storage tank can be overlaid with the name of the tank contents, along with a graphical and numerical representation of how full the storage tank is. The element data can also comprise visual overlays that show the state of valves and the direction and quantity of feed stock and product flows. Through gestural and user' keyboard/mouse input methods, the user can make control decisions regarding the physical process. The system may also have security to ensure only those with permission would be allowed to make changes. Security and authorization settings can also limit what information is displayed in the ARHCS. For example, a plant visitor/contractor may not be able to view proprietary process information.

The ARHCS can provide geo-located permissions. A technician working on a specific system who is physically co-located with that system may be granted a higher level of permission to start, stop, reset or interact with a physical process. An overhead or map view aiding the user in navigation can be provided through a large industrial plant. Additionally, the user can access overlays that map the material flows in the industrial process. The element data can comprise safety information related to hazardous materials or locations and/or provide information on safe practices for dangers. The ARHCS can provide person-to-person collaboration with audio and visual communication via directly on the task. Collaboration generally works well for regular work flow but also for safety. Workers can signal each other when conditions to proceed are safe. For example, when a lockout has been completed, or the opposite, that safe starting has commenced after a lockout has been removed.

FIG. 2 is a block diagram representation of an example ARHCS 200 including a wireless transceiver 216 coupled to an antenna 219 that supports wireless communications which can be integrated into a disclosed head mounted combination for a user. As known in the art of communications, transceiver 216 includes a receive chain and a transmit chain, with in the receive chain amplifier(s), filter(s) and an Analog-to-Digital Converter (ADC), and in the transmit chain a Digital-to-Analog Converter (DAC), filter(s) and an amplifier.

ARHCS 200 includes a pair of displays 201a and 201b configured to be embedded in or on an inside surface of the eye shield or in or on lenses under the eye shield of the head mounted combination. The displays are coupled to a processor 212 which controls a display driver (not shown) to generate computer-generated imagery (CGI) in the displays 201a and 201b. It can also be a new kind of eye shield with the display mirror for the projection being just part of the safety equipment usable in the traditional way without the ARHCS as well as with. Display 201a is shown embedded in lens 226a and display 201b is shown embedded within lens 226b. The AR provided by ARHCS 200 can combine a real-world view with CGI by projecting the CGI through a partially reflective mirror and viewing the real world directly (optical see-through) or electronically by accepting real-world video from a camera and mixing it electronically with the CGI (video see-through).

The ARHCS 200 includes an outer seal 240 used to prevent water and gas ingress, particulate (i.e. dust, vapors) penetration and to prevent generating a spark that can cause ignition in flammable atmospheres for making the ARHCS safe to use in a hazardous environment. The material for the outer seal 240 can depend on the environment. Silicones, acrylonitrile butadiene rubber (NBR), poly-tetra-fluoroethylene (PTFE, also known as TEFLON) are all examples of possible seal materials. The outer seal 240 can enable certification for use in flammable or explosive atmospheres.

There may also be another seal for protecting the users' face and eyes. The user's eyes and face are generally protected for health and safety reasons, but the level of protection needed is highly variable depending on the environment and different options may be present in different versions of the head mounted combination. The outer seal 240 can be installed as an o-ring between two or more parts of the enclosure of the hardware. Any interface ports (i.e. for charging or software updates) can be similarly sealed, or else covered by a screw-down or clamp down covered port sealed with its own O-ring. This feature allows the ARHCS to be recharged or updated when it is no longer within the hazardous environment.

The ARHCS 200 is shown including a data bus 202 for communicating information data, signals, and information between various components of ARHCS 200. ARHCS 200 components include an input/output (I/O) device 204 that processes a user's action, such as selecting keys from a virtual keypad/keyboard, for example a Bluetooth-like keyboard, selecting one or more buttons or links, and sends a corresponding signal to the data bus 202. The displays 201a, 201b are generally mounted a short distance in front of the user's eyes. ARHCS 200 includes an input control such as a cursor control 213 (e.g., a virtual keyboard, virtual keypad, or a virtual mouse). ARHCS 200 also includes a location sensor 221 such as a Global Positioning System (GPS), orientation sensor 222 such as including accelerometer, gyroscope and magnetometer, and a gaze sensor 223 such as comprising cameras or other optical sensors. An auxiliary sensor 224 is shown that can be for gas sensing that may be a wired or wireless sensor. An optional audio IO component 205 may also to allow the user to hear audio. One embodiment uses a vibrator as in most cell phones for immediate alerts but also for safety to “wake” a user if the orientation and body sensors (like a blood O₂ sensor) find they may have become unresponsive.

The transceiver 216 uses antenna 219 to transmit and receive signals between the ARHCS 200 and other devices or systems, such as another user device, or another network computing device (e.g., a server) via a communication link to a wireless network. The processor 212, which can be a microcontroller, digital signal processor (DSP), microcontroller unit (MCU) or other processor, processes these various signals, such as for display on the display(s) 201a, 201b of the ARHCS 200 or transmission to other devices via a communication link. Processor 212 may also control transmission of information.

ARHCS 200 also includes a system memory 214 (e.g., random access memory (RAM)), and/or a hard drive 217 or other persistent storage device. ARHCS 200 performs specific operations by the processor 212 and other components by executing one or more sequences of instructions contained in system memory 214. Logic may be encoded in a computer readable medium. In one embodiment, the logic is encoded in a non-transitory computer readable medium. Execution of instruction sequences may be performed by the processor 212.

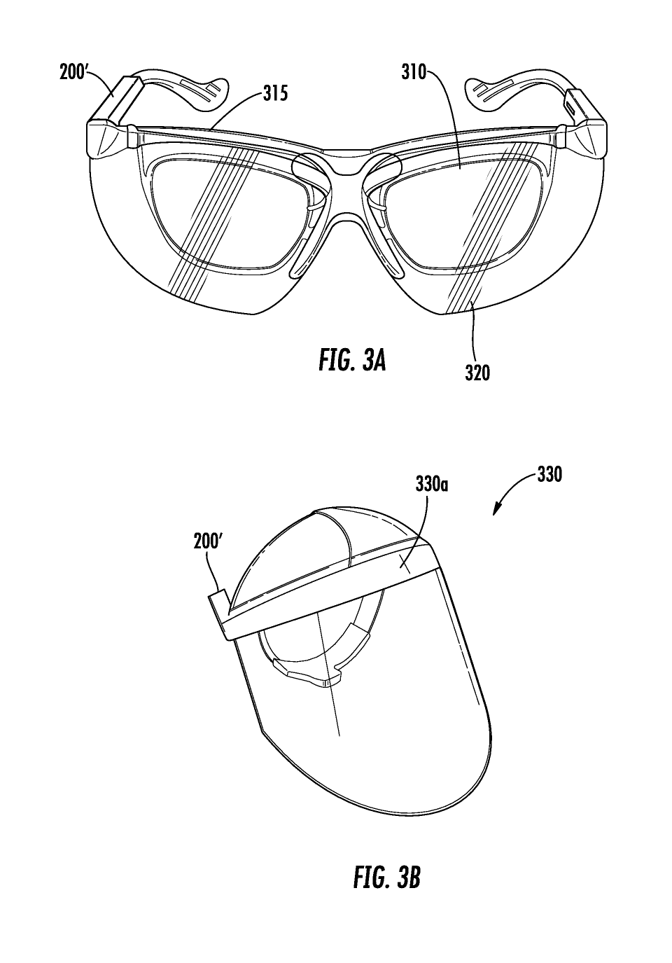

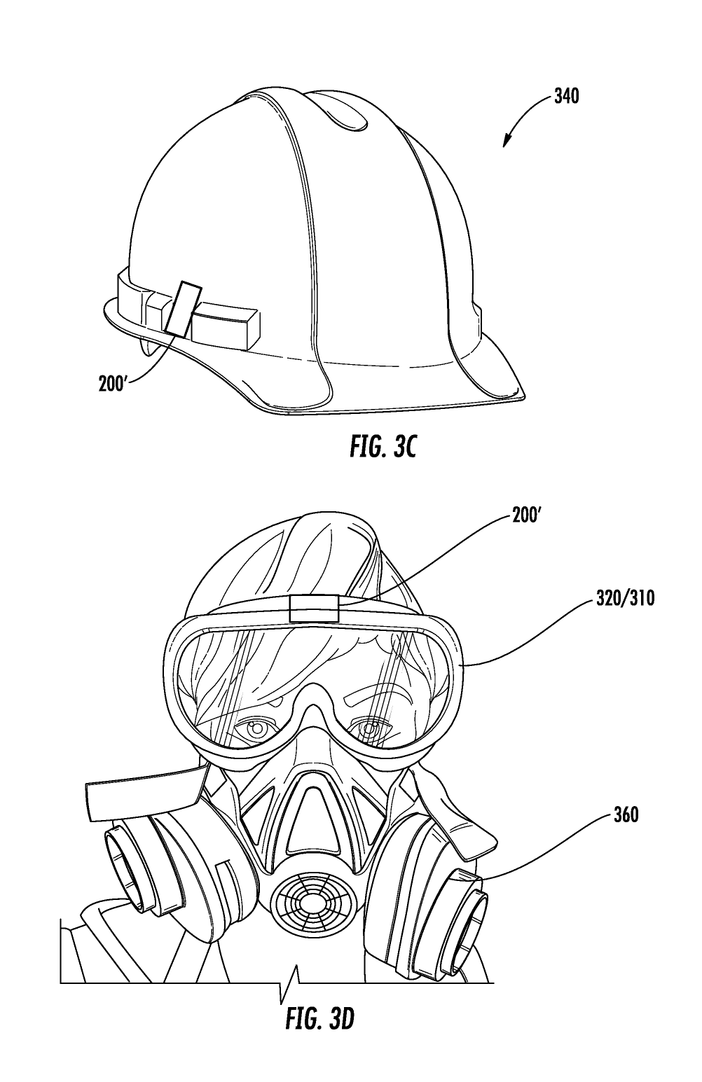

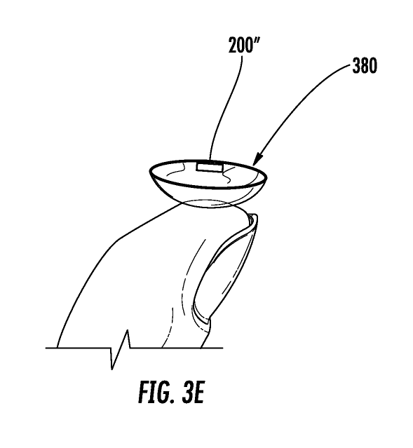

FIGS. 3A-3E show example head mounted combinations configured for being secured to a head of the user including at least an eye shield and an ARHCS 200. Since the head mounted combinations generally always have a lens (226a, 226b) for real world viewing and a display (201a, 201b) for computer generated information, the ARHCS 200 is attached to the user to overlay vision in all cases and the lens and display is generally positioned somewhere in front of the eyes to be in the user's field of view.

The processor portion of the ARHCS 200 can be part of the edge or mount of the lens (like a thick frame or thick arm of glasses), or it can be attached such as by wire to a pack that mounts on the back or side of a helmet. The wire could also be longer and clip to the frame body. There can also be a wireless option where the lens has its own power supply (e.g., a battery) and a small size processing unit to just display from the main processing pack. Cursor control 213 can generally be mounted anywhere accessible to the user. The audio I/O 205 can be positioned near enough to the ears to be audible, such as earbuds compatible with hearing protection or built into the hearing protection itself.

FIG. 3A shows an example head mounted combination comprising safety glasses 310 including an example ARHCS shown as ARHCS 200′ (in FIG. 3A, as well as FIGS. 3B-3E described below) because its the lens (226a, 226b) and display (201a, 201b) are remotely located from the rest of the ARHCS (wired or wireless connection) being in the field of view of the user's eye. The ARHCS 200′ is shown attached to the frame 315. In this case, the materials and construction of the head mounted combination can meet impact resistance standards and the safety glasses 310 will generally be shaped to prevent particles from reaching the eye of the user from the sides and from below. A replaceable (removable) eye shield 320 is also shown which can be clipped onto the glasses 310, as normal safety glasses are generally discarded if they become damaged in normal use to avoid the need to throw out the entire head mounted combination, where the eye shield 320 that does the bulk of the user protection function is thus replaceable and can be sold as a consumable. The eye shield 320 surrounds and protects both the user and the ARHCS 200 from impacts and can comprise polycarbonate or a similar polymer material. The head mounted display portion is not much like safety glasses, and it would have a protective shield that is either part of the ARHCS outright, or else it goes over the ARHCS.

FIG. 3B shows an example head mounted combination comprising a face shield 330 including an ARHCS with ARHCS 200′ shown. The ARHCS 200′ is shown on an inside surface of the face shield 330. The ARHCS 200′ is shown attached to the helmet portion 330a of the face shield. The face shield 330 can be a removable face shield being clipped on the top of an eye shield. Optionally, instead of the replaceable shield 320 as described above, a large face shield could be attached to the front of the ARHCS 200 for environments that need face protection too besides user eye protection. Similarly, the face shield version will generally have the face shield as something that snaps on to the ARHCS, rather than being worn under an existing face shield.

FIG. 3C shows an example head mounted combination comprising a hard hat 340 including an example ARHCS with ARHCS 200′ shown. The ARHCS 200′ is shown positioned in a groove on an outside side surface of the hard hat 340. The ARHCS is configured to be worn with a standard hardhat, or can be integrated into a custom hard hat without causing the user discomfort or reducing the protection provided by the hard hat.

FIG. 3D shows an example head mounted combination comprising a respirator mask 360 including an example ARHCS 200 with ARHCS 200′ shown. The ARHCS is built into the top of the safety glasses 310 over the respirator mask 360. Above the respirator mask 360 the user is wearing the replaceable eye shield 320 over the safety glasses 310 shown in FIG. 3A now shown as 320/310. The respirator mask 360 can be used in confined space entry, fire crews, or access to environments that do not provide a safe atmosphere.

FIG. 3E shows an example head mounted combination comprising a screen 380 resembling a contact lens including an example ARHCS shown as ARHCS 200″ shown adapted to be worn in contact with an eye of the user. ARHCS 200″ due to size and access limitations being on the user's eye generally lacks several components included with ARHCS 200′. The display in this embodiment is embedded near the center of the screen within the screen to provide AR functionality which is wirelessly coupled to a small processor embedded in the periphery of the lens. An embedded lens when customized to the user can also correct a user's eyesight.

### EXAMPLE

To test a disclosed head mounted combination an extended version of the Matrikon Unified Architecture (UA) Software Development toolkit that interfaces with a Microsoft HOLOLENS AR headset device was configured together. The toolkit provided sample code, templates for visual CGI overlays, and for constructing the location identification tags and database. The database was made available as UA metadata, to be advertised by the OLE for Process Control (OPC) UA (OPC UA) by a server acting as the data source or gateway. Client software installed on the HOLOLENS ARHCS was responsible for consuming both the location database and the process information to display the required information on the respective displays of the head mounted combination.

Disclosed embodiments are further illustrated by the following specific Examples, which should not be construed as limiting the scope or content of this Disclosure in any way. While various disclosed embodiments have been described above, it should be understood that they have been presented by way of example only, and not limitation. Numerous changes to the subject matter disclosed herein can be made in accordance with this Disclosure without departing from the spirit or scope of this Disclosure. For example, with satellite communications which are now technically feasible disclosed methods can also be applied to remote workers working on projects such as pipelines or in forestry, and also for shipping and shipping receiving. In addition, while a particular feature may have been disclosed with respect to only one of several implementations, such feature may be combined with one or more other features of the other implementations as may be desired and advantageous for any given or particular application.

As will be appreciated by one skilled in the art, the subject matter disclosed herein may be embodied as a system, method or computer program product. Accordingly, this Disclosure can take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, this Disclosure may take the form of a computer program product embodied in any tangible medium of expression having computer usable program code embodied in the medium.

1. A method of protecting and assisting a user in an industrial facility, comprising:
providing head mounted combination configured for being secured to a head of said user including at least an eye shield and an augmented reality headset computer system (ARHCS) providing communications for communicating with an industrial data management system over a wireless communication channel including a processor, system memory, transceiver and antenna, a location sensor, orientation sensor, and a gaze sensor, at least one display embedded in or on an inside surface of said eye shield or in or on lenses under said eye shield that are coupled to said processor, and client software stored in said system memory, said client software implementing:
determining what said user is looking at;
from what said user is looking at and a 3D model of system elements in said industrial facility overlaying computer generated representations of viewed ones of said system elements that are within a field of view to a real world view of said user;
adding at least one display marker to said viewed ones of said system elements which have further data available including a first display marker to a first system element which has available first element data to indicate said further data is available, and
responsive to said user triggering said first display marker, displaying said first element data in said display for viewing by said user.

2. The method of claim 1, wherein said determining what said user is looking at is determined from a location of said user, said field of view of said user, and a gaze of said user.

3. The method of claim 1, wherein said first element data comprises real-time process data obtained from said system elements.

4. The method of claim 1, wherein said 3D model is stored in said system memory.

5. The method of claim 2, wherein said overlaying computer generated representations of viewed ones of said system elements comprises mathematically projecting what said user is looking at, said location of said user, and an orientation of said head mounted combination all into said 3D model.

6. The method of claim 2, wherein said determining said gaze of said user comprises identifying when said user is intentionally looking at a particular one of said display markers for a predetermined period of time.

7. The method of claim 1, wherein said ARHCS further comprises an outer seal surrounding electronics of said ARHCS including said processor, said system memory, said transceiver, said location sensor, said orientation sensor, and said gaze sensor for preventing water or gas ingress.

8. A head mounted combination for protecting and assisting a user in an industrial facility, comprising:
at least an eye shield and an augmented reality headset computer system (ARHCS) providing communications for communicating with an industrial data management system over a wireless communication channel including a processor, system memory, transceiver and antenna, a location sensor, orientation sensor, and a gaze sensor, at least one display embedded in or on an inside surface of said eye shield or in or on lenses under said eye shield that are coupled to said processor, and client software stored in said system memory, said client software implementing:
determining what said user is looking at;
from what said user is looking at and a 3D model of system elements in said industrial facility overlaying computer generated representations of viewed ones of said system elements that are within a field of view to a real world view of said user;
adding at least one display marker to said viewed ones of said system elements which have further data available including a first display marker to a first system element which has available first element data to indicate said further data is available, and
responsive to said user triggering said first display marker, displaying said first element data in said display for viewing by said user.

9. The head mounted combination of claim 8, wherein said determining what said user is looking at is determined from a location of said user, said field of view of said user, and a gaze of said user.

10. The head mounted combination of claim 8, wherein said first element data comprises real-time process data obtained from said system elements.

11. The head mounted combination of claim 8, wherein said 3D model is stored in said system memory.

12. The head mounted combination of claim 9, wherein said overlaying computer generated representations of viewed ones of said system elements comprises mathematically projecting what said user is looking at, said location of said user, and an orientation of said head mounted combination all into said 3D model.

13. The head mounted combination of claim 9, wherein said determining said gaze of said user comprises identifying when said user is intentionally looking at a particular one of said display markers for a predetermined period of time.

14. The head mounted combination of claim 8, wherein said ARHCS further comprises an outer seal surrounding electronics of said ARHCS including said processor, said system memory, said transceiver, said location sensor, said orientation sensor, and said gaze sensor for preventing water or gas ingress.
